(19) United States
(12) Patent Application Publication
(10) Pub. No.: US 2018/0271783 A1
(43) Pub. Date: Sep. 27, 2018

(54) REPEATED ADMINISTRATION OF LENTIVIRAL VECTORS TO RESPIRATORY CELLS

(71) Applicant: UNIVERSITY OF IOWA RESEARCH FOUNDATION, Iowa City, IA (US)

(72) Inventors: Paul B. McCray, Iowa City, IA (US); Patrick L. Sinn, Iowa City, IA (US)

(73) Assignee: UNIVERSITY OF IOWA RESEARCH FOUNDATION, Iowa City, IA (US)

(21) Appl. No.: 15/977,858

(22) Filed: May 11, 2018

Related U.S. Application Data
(63) Continuation of application No. 12/433,659, filed on Apr. 30, 2009, now abandoned.
(60) Provisional application No. 61/049,175, filed on Apr. 30, 2008.

(51) Int. Cl. A61K 9/00; A61K 48/00

(52) U.S. Cl. CPC A61K 9/0073; A61K 48/005; A61K 48/0075; A61K 48/0083; A61K 9/0043; C12N 2740/15043; C12N 2740/15045; C12N 2810/6009; C12N 2810/6018; C12N 2810/6081; C07K 2319/01

(57) ABSTRACT

The invention provides methods for administering lentiviral vectors to the respiratory system of a patient to treat a disease.

### RELATED APPLICATION(S)

This application is a continuation application of U.S. application Ser. No. 12/433,659, filed Apr. 30, 2009, which claims the benefit of priority of U.S. application Ser. No. 61/049,175, filed Apr. 30, 2008, which applications are herein incorporated by reference.

### STATEMENT OF GOVERNMENT SUPPORT

This invention was made with government support under K01 DK-073367, R01 HL-075363 and PO1 HL-51670 awarded by the National Institutes of Health. The government has certain rights in the invention.

### BACKGROUND

Several problems limit the application of gene transfer as a tool for pulmonary cell biology studies and impede its use for treating diseases of the respiratory system. Mucosal innate and adaptive immune responses against the vector or vector-encoded proteins represent a significant impediment to clinical applications and are well documented for viral vectors such as adenovirus (Ad) (Harvey et al., Mol Ther, 3, 206-215 (2001) and Harvey et al., J Clin Invest, 104, 1245-1255 (1999)) and adeno-associated virus (AAV) (Halbert et al., Hum Gene Ther, 17, 440-447 (2006)). Indeed, a driving force behind the development of helper-dependent adenoviral vectors (Koehler et al., Gene Ther, 13, 773-780 (2006)) and the search for alternative AAV vector capsids (Gao et al., Curr Gene Ther, 5, 285-297 (2005) and Limberis et al., PNAS, 103, 12993-12998 (2006)) has been avoidance of adaptive immune responses. An alternative strategy is the use of integrating viral vectors of the retrovirus family (Goldman et al. Hum Gene Ther, 8, 2261-2268 (1997) and Sinn et al., Gene Ther, 12, 1089-1098 (2005)). Thus, a major limitation for gene transfer is the inability to re-administer vectors, e.g., as transgene expression wanes, and methods for such administration are needed.

### SUMMARY OF CERTAIN EMBODIMENTS OF THE INVENTION

The efficacy of repeatedly administering lentiviral vectors to cells of the respiratory system is described herein. Using quantitative bioluminescent imaging, it was discovered that consecutive daily dosing achieved a linear increase in gene expression and greatly increased the number of epithelial cells targeted. Surprisingly, reporter gene expression also increased additively following each of 7 doses of feline immunodeficiency virus (FIV) delivered over consecutive weeks at 1 dose/week, without the development of systemic or local neutralizing antibodies. This approach enhanced expression of both reporter and therapeutic transgenes. Transduction efficiency achieved following a single dose of FIV expressing mouse erythropoietin was insufficient to increase hematocrit, whereas 7 consecutive daily doses significantly increased hematocrit. These unexpected results contrast strikingly with findings reported for adenoviral and AAV vectors and represent a significant advance for the applications of lentiviral vectors in respiratory cell biology and gene transfer.

Accordingly, certain embodiments of the present invention provide methods for treating a patient (e.g., a human patient), comprising administering a lentiviral vector that comprises a nucleotide sequence encoding a therapeutic protein to a tissue of the respiratory system of the patient, wherein the administration comprises administering the lentiviral vector in at least two consecutive dosages, wherein two consecutive dosages of the administration are separated by an interval of more than one day. While two consecutive dosages of the treatment are separated by an interval of more than one day (e.g., about 2, 3, 4, 5 or 6 days; about 1, 2, 3, 4, 5, 6, 7, 8, 9 or 10 weeks; or about 1, 2, 3, 4, 5, 6, 7, 8, 9, 10, 11 or 12 months), other dosages within the treatment regimen may be separated by an interval of about one day, of more than about one day, and/or of less than about one day.

In certain embodiments, the lentiviral vector is a human immunodeficiency viral vector, a visna-maedi virus viral vector, a caprine arthritis-encephalitis virus viral vector, an equine infectious anemia virus viral vector, a feline immunodeficiency virus (FIV) viral vector; bovine immune deficiency virus (BIV) viral vector, a simian immunodeficiency virus (SIV) viral vector, a murine Moloney leukemia virus viral vector, a foamy virus viral vector, or an avian leukosis virus viral vector.

In certain embodiments, the lentiviral vector is a FIV viral vector.

In certain embodiments, the lentiviral vector is pseudotyped with an envelope glycoprotein.

In certain embodiments, the lentiviral vector is pseudotyped with a filovirus, coronavirus, or influenza envelope glycoprotein.

In certain embodiments, the envelope glycoprotein is glycoprotein-64 (GP64).

In certain embodiments, the envelope glycoprotein is an Autographa californica multinuclear polyhedrosis virus (AcMNPV) glycoprotein.

In certain embodiments, the therapeutic protein is cystic fibrosis transmembrane regulator protein (CFTR), Alpha 1 antitrypsin, ATP-binding cassette A3 protein (ABCA3), surfactant protein B (SFTPB) or surfactant protein C (SFTPC).

In certain embodiments, the therapeutic protein is CFTR.

In certain embodiments, the tissue of the respiratory system comprises airway epithelial cells.

In certain embodiments, the tissue of the respiratory system is lung tissue, nasal tissue, tracheal tissue, bronchial tissue, bronchiolar or alveolar epithelial cell tissue.

In certain embodiments, the administration comprises administering at least three doses, e.g., 4, 5, 6, 7, 8, 9, 10 or more, e.g., 10-20 doses.

In certain embodiments, the two consecutive dosages of the administration are separated by an interval of about one week.

In certain embodiments, the two consecutive dosages of the administration are separated by an interval of about one month.

In certain embodiments, the administration is via aerosol, dry powder, bronchoscopic instillation, or intra-airway (tracheal or bronchial) aerosol.

In certain embodiments, the patient has been diagnosed with cystic fibrosis.

### BRIEF DESCRIPTION OF THE FIGURES

FIG. 1. Acute Repeat Administration of Lentiviral Vector Expressing Luciferase (Luc). GP64 pseudotyped FIV expressing Luc was delivered to mice following the protocol as shown (A). At the indicated time points, mice were given luciferin via I.P. injection and photographed using bioluminescent imaging (B). At the conclusion of the experimental protocol, mice received a boost dose of vector. One week later, serum and BAL was collected. Total serum and BAL IgG antibodies against GP64 were determined by ELISA (C). n=5; *p<0.0001 compared to naive control by ANOVA, using Tukey-Kramer adjustment for multiple comparisons.

FIG. 2. Acute Repeat Administration of Lentiviral Vector Expressing β-gal. GP64 pseudotyped FIV expressing nuclear targeted β-gal was delivered to mice following the same protocol as FIG. 1A. Four weeks following the final dose, sections were collected from 5 standardized levels based on anatomical landmarks, as shown schematically (A). One level is ˜30 microns. Low power photographs (B) of both olfactory (C) and respiratory (D) epithelia were collected from each level. Using ImagePro software, the percent of β-gal positive surface epithelial cells was determined for both olfactory (E) and respiratory (F) epithelia. In addition, the total percent positive was determined as a function of number of doses (G). n=3; *p<1×10⁻⁵, F-test for dependence on dose response.

FIG. 3. Priming Vector Doses Followed by a Test Dose. Priming or test doses of GP64 pseudotyped FIV, Ad5, or VSV-G pseudotyped FIV was delivered to mice at two week intervals following the protocol as shown (A). At the indicated time points, mice that received GP64-FIV (B), Ad5 (C), or VSV-G-FIV (D) were given luciferin via I.P. injection and photographed using bioluminescent imaging. Mice were imaged and bled between each dose of vector as indicated. At the conclusion of the experimental protocol, mice received a boost dose of vector. One week later, serum and BAL was collected. Total IgG GP64 or Ad antibodies were quantified in sera (E) and BAL (F) by ELISA. n=4; *p<0.001 compared to naive control by ANOVA, using Tukey-Kramer adjustment for multiple comparisons. Inactivating antibodies against Ad (grey circles) or GP64 (black diamonds) were measured in the BAL (G). Equal transducing units of vector (1.25×10⁷ TU, as determined by real-time PCR) were delivered for priming and test doses.

FIG. 4. Re Administration of Lentiviral Vector Versus Baculoviral Vector. Three doses of GP64 pseudotyped FIV or baculovirus were delivered to mice at two week intervals. Mice received either nasal instilled or tail vein (I.V.) delivered vector. Total IgG anti-GP64 antibodies were quantified in sera collected one week following the final dose by ELISA (A). Inactivating antibodies against GP64-FIV-Luciferase in the sera from each experimental group were determined (B). n=4.

FIG. 5. Chronic Repeat Administration of Lentiviral Vector. GP64 pseudotyped FIV or Ad5 expressing Luc was delivered at 1 week intervals to mice following the protocol as shown (A). At the indicated time points, mice that received GP64-FIV-Luc (B) or Ad5-Luc (C) were given luciferin via I.P. injection and photographed using bioluminescent imaging. Between each dose of vector, mice were imaged and bled as indicated. n=5; *p<0.0001, **p<0.001 compared to naive control by ANOVA, using Tukey-Kramer adjustment for multiple comparisons.

FIG. 6. Adaptive Immune Response Following Chronic Repeat Administration of Lentiviral Vector. Mice were bled between each weekly dose during the chronic repeat administration experimental protocol. Total IgG serum antibodies against GP64 or Ad were determined by ELISA (A). At the conclusion of the chronic repeat administration experimental protocol, mice received a boost dose of vector. One week later, serum and BAL was collected. Total serum and BAL IgG antibodies (B) or BAL IgA antibodies (C) against GP64 or Ad were determined by ELISA. The presence of neutralizing antibodies against GP64-FIV-Luciferas or Ad5-Luc in the BAL was determined (D). n=4 or 5.

FIG. 7. Chronic Repeat Administration of Lentiviral Vector+/−an Internal Promoter. GP64-FIV-luciferase was delivered at 1 week intervals to mice following the protocol as shown (A). Between each dose of vector, mice were imaged. At the indicated time points were given luciferin via I.P. injection and photographed using bioluminescent imaging (B). Long-term persistence (˜1.5 years) was followed in two cohorts of mice, group A and group E (C). n=5.

FIG. 8. Repeat Administration of a Lentiviral Vector Expressing a Secreted Protein. A single nasal instilled dose of GP64-FIV-mEPO (squares) was compared to 7 doses (1 dose/day) delivered over 7 consecutive days (diamonds). Hematocrit was determined as an indirect measure of mEPO expression. n=5; *p<0.001 compared to naive control (circles) by ANOVA, using Tukey-Kramer adjustment for multiple comparisons.

FIG. 9. Acute Repeat Tracheal Administration of Lentiviral Vector Expressing Luciferase. GP64 pseudotyped FIV expressing luciferase (1.25×10⁷ TU) was delivered to mice via tracheal intubation following the protocol as shown (A). Mice receiving the indicated number of doses were given luciferin via I.P. injection and photographed using bioluminescent imaging (B). The persistence of expression was determined for 12 weeks in both the lung and nose (C). At the conclusion of the experimental protocol, mice received a boost dose of vector. One week later, serum and BAL was collected. Total serum and BAL antibodies against GP64 were determined by ELISA (D). The absence of neutralizing antibodies was verified in the BAL (E). n=4.

### DETAILED DESCRIPTION

Local and systemic immune responses to adenoviral and AAV vectors effectively prevent their repeated pulmonary administration. As described herein, lentiviral vectors were successfully re-administered in vivo to respiratory epithelia, which re-administration caused increased expression of both reporter genes and a therapeutic gene. The FIV lentivirus was delivered at intervals of days or weeks with associated stable increases in reporter or therapeutic transgene expression. Unexpectedly, despite repeated vector application and low level antibody production, blocking immune responses failed to develop. This is the first demonstration of retroviral vector re-administration at a mucosal surface.

Thus, reported here for the first time is the successful in vivo re-administration of lentiviral vectors to respiratory epithelia to increase expression of both reporter genes and a therapeutic gene. Unlike the well-characterized and widely recognized local and systemic immune responses to adenoviral vectors that may prevent repeat pulmonary administration, little is known regarding host responses to lentiviral vectors. The FIV lentivirus was delivered at intervals of days or weeks with associated stable increases in reporter or therapeutic transgene expression. Despite repeated vector application and low level antibody production, blocking immune responses failed to develop.

### Immune Responses

Innate immune responses to viruses and viral vectors are a first line of defense generated within minutes to hours following viral vector administration and contrast with the slower to develop adaptive immunity. These responses have been extensively studied with the adenoviral and AAV vectors under investigation for pulmonary gene transfer applications. Many preclinical studies and clinical trials with adenoviral vectors document increases in systemic chemokines and proinflammatory cytokines including RANTES, IP-10, MIP-2, IFN-γ, TNF-α, IL-6, IL-10 and IL-12 in humans and mice. Further, a priming dose of an adenoviral vector will elicit sufficient humoral and cell-mediated immunity to prevent pulmonary re-administration without the use of immunosuppressive agents or the masking of vector epitopes with formulations such as pegylation. Similarly, a second dose of AAV typically yields lower levels of pulmonary gene transfer because of neutralizing antibody responses. Pre-existing immunity against either Ad or AAV may also present a significant barrier to initial use of these vectors.

As described herein, minimal innate and adaptive immune responses were observed following topical delivery of a GP64 pseudotyped FIV vector to murine airway epithelia. Repeated administration of the FIV vector failed to elicit immune responses that would prevent re-administration even after 7 weekly doses. It is unlikely that pre-existing immunity to the GP64 envelope glycoprotein will present a barrier.

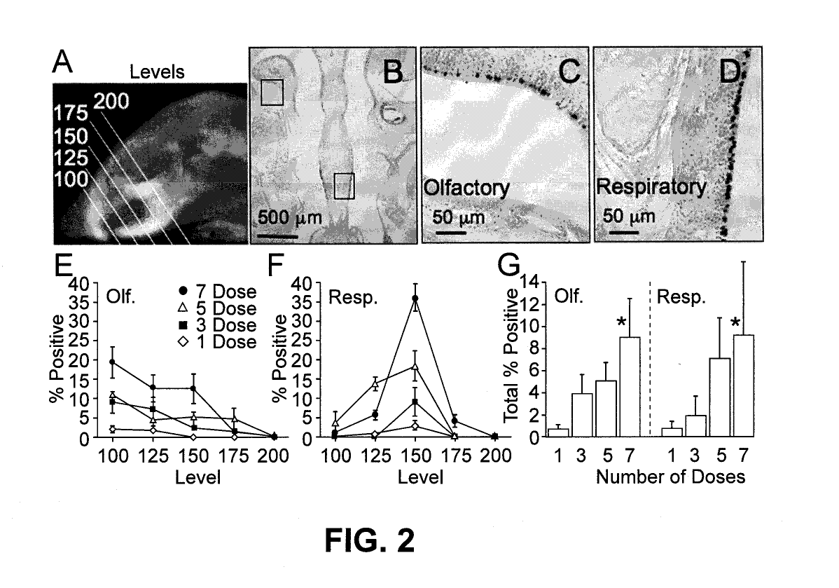

The GP64 pseudotyped FIV vector efficiently transduced respiratory epithelia and persistently expressed a transgene. Additive increases in transgene expression were observed with repeat dosing. It is believed that this increase in expression represents both an increase in the percentage of cells expressing a transgene (see FIG. 2) and an increase in the number of transgene copies/cell. The lack of evidence for development of immune responses to the reporter genes Luc or β-gal was surprising. For example, Naldini and colleagues recently reported innate and adaptive immune responses to lentiviral vectors administered systemically and targeting hepatocytes in mouse models (Brown et al., Blood, 109, 2797-2805 (2007)). The results presented herein indicate that respiratory mucosal application of a lentivirus vector unexpectedly is less immunostimulatory than systemic delivery.

### Gene Transfer to the Respiratory Tract

For genetic diseases, the expression of a therapeutic protein over the life of the affected individual is a goal. For non-integrating vector platforms, repeated administration will be important, and this aspect of treatment presents limitations. The re-administration of lentiviral vectors to the respiratory tract offers the possibility to increase the overall transduction efficiency. For example, sequential vector administration to individual lung lobes may be advantageous for practical and safety reasons. In addition, the ability to successfully re-dose provides a therapeutic option should expression wane over time. The findings presented herein have important implications for the use of lentiviral gene transfer technology in tools for investigating respiratory cell biology and for the development of pulmonary disease therapies.

Cystic fibrosis is a respiratory disease caused by a genetic mutation of a single mutated gene (i.e., the cystic fibrosis transmembrane conductance regulator (CFTR) gene that encodes the protein CFTR). Another genetic respiratory disease is alpha-1-antitrypsin deficiency, caused by a mutation in the SERPIN A1 gene. Other diseases include disorders (e.g., hereditary disorders) of surfactant metabolism. Disorders of surfactant metabolism can be caused by mutations in the ATP-binding cassette A3 protein (ABCA3), surfactant protein B (SFTPB) or surfactant protein C (SFTPC). These diseases could be better treated (e.g., controlled or cured) with improved gene therapy. For this to occur, advancements in gene therapy technology, such as increased transduction efficiency, increased levels of transgene expression, and increased length of transgene expression are important. It would be beneficial to be able to repeatedly administer a functional gene to compensate for the mutated gene(s) that cause a disease. Accordingly, certain embodiments of the present invention provide methods of treating diseases by administering (e.g., repeatedly administering) a vector that includes a nucleic acid sequence that encodes a protein that treats the disease.

The methods described herein may provide for increased numbers of transduced cells, increased and sustained transgene expression, increased expression level of transgene, increased length of transgene expression, increased likelihood for gene therapy success, and decreased immune response to gene therapy. Thus, the methods described herein allow for repeated dosing of the transgene, which provides an increased likelihood for gene therapy success.

### Retroviruses; Retroviral Vectors

The term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules that encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. There are several genera included within the family Retroviridae, including Cisternavirus A, Oncovirus A, Oncovirus B, Oncovirus C, Oncovirus D, Lentivirus, and Spumavirus. Retroviruses infect a wide variety of species, and may be transmitted both horizontally and vertically. They can be integrated into the host DNA, and are capable of transmitting sequences of host DNA from cell to cell. This has led to the development of retroviruses as vectors for various purposes including gene therapy.

Retroviruses, including human foamy virus (HFV) and human immunodeficiency virus (HIV) have gained much recent attention, as their target cells are not limited to dividing cells and their restricted host cell tropism can be readily expanded via pseudotyping with vesicular stomatitis virus G (VSV-G) envelope glycoproteins.

Vector systems generally have a DNA vector containing a portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle assembly.

For cells that are naturally dividing or are stimulated to divide by growth factors, simple retroviruses like murine leukemia virus (MLV) vectors are suitable delivery systems. A major limitation in the use of many commonly used retroviral vectors in gene transfer, however, is that many of the vectors are restricted to dividing cells. If a non-dividing cell is the target cell, then a lentivirus, which is capable of infecting non-dividing cells, may be used.

As used herein, the term “lentivirus” refers to a group (i.e., a genus) of retroviruses that give rise to slowly developing disease. Viruses included within this group include HIV (human immunodeficiency virus; including HIV type 1, and HIV type 2); visna-maedi; the caprine arthritis-encephalitis virus; equine infectious anemia virus; feline immunodeficiency virus (FIV); bovine immune deficiency virus (BIV); and simian immunodeficiency virus (SIV).

Lentiviruses including HIV, SIV, FIV and equine infectious anemia virus (EIAV) use several viral regulatory genes in addition to the simple structural gag-pol-env genes for efficient intracellular replication. Thus, lentiviruses use more complex strategies than classical retroviruses for gene regulation and viral replication, with the packaging signals apparently spreading across the entire viral genome. These additional genes display a web of regulatory functions during the lentiviral life cycle.

A “source” or “original” retrovirus is a wild-type retrovirus from which a retrovirus, e.g., a pseudotyped retrovirus, is derived, or is used as a starting point, during construction of the packaging or transgene vector, for the preparation of one or more of the genetic elements of the vector. The genetic element may be employed unchanged, or it may be mutated (but not beyond the point where it lacks a statistically significant sequence similarity to the original element). A vector may have more than one source retrovirus, and the different source retroviruses may be, e.g., MLV, FIV, HIV-1 and HIV-2, or HIV and SIV. The term “genetic element” includes but is not limited to a gene.

The term “replication-competent” refers to a wild-type virus or mutant virus that is capable of replication, such that replication of the virus in an infected cell result in the production of infectious virions that, after infecting another, previously uninfected cell, causes the latter cell to likewise produce such infectious virions. The present invention contemplates the use of a replication-defective virus.

As used herein, the term “attenuated virus” refers to any virus (e.g., an attenuated lentivirus) that has been modified so that its pathogenicity in the intended subject is substantially reduced. The virus may be attenuated to the point it is nonpathogenic from a clinical standpoint, i.e., that subjects exposed to the virus do not exhibit a statistically significant increased level of pathology relative to control subjects.

The present invention contemplates the use of a modified retrovirus, e.g., a modified lentivirus, e.g., a modified FIV. In some embodiments, the retrovirus is an mutant of murine leukemia virus, human immunodefciency virus type 1, human immunodeficiency virus type 2, feline immunodeficiency virus, simian immunodeficiency virus, visna-maedi, caprine arthritis-encephalitis virus, equine infectious anemia virus, and bovine immune deficiency virus, or a virus comprised of portions of more than one retroviral species (e.g., a hybrid, comprised of portions of MLV, FIV, HIV-1 and HIV-2, or HIV-1 and/or SIV).

A reference virus is a virus whose genome is used in describing the components of a mutant virus. For example, a particular genetic element of the mutant virus may be said to differ from the cognate element of the reference virus by various substitutions, deletions or insertions. It is not necessary that the mutant virus actually be derived from the reference virus.

Certain embodiments of the invention relate to the use of FIV. A reference FIV sequence is found in Talbott et al., PNAS, 86, 5743-5747 (1989); Genbank access# NC 001482. In certain embodiments, a three-plasmid transient transfection method can be used to produce replication incompetent pseudotyped retrovirues (e.g., FIV). General methods are described in Wang et al., J Clin Invest, 104, R55-62 (1999) and Johnston et al., J Virol, 73, 4991-5000 (1999).

### Retroviral Vector System

The vectors used in connection with certain embodiments of the present invention may be derived from a retrovirus (e.g., a lentivirus, e.g., FIV). Retrovirus vectors allow (1) transfection of the packaging vectors and envelope vectors into the host cell to form a packaging cell line that produces essentially packaging-vector-RNA-free viral particles, (2) transfection of the transgene vector into the packaging cell line, (3) the packaging of the transgene vector RNA by the packaging cell line into infectious viral particles, and (4) the administration of the particles to target cells so that such cells are transduced and subsequently express a transgene.

The packaging vectors and transgene vectors can generate replication-incompetent viruses.

The envelope protein can in certain embodiments be a retroviral envelope, a synthetic or chimeric envelope, or the envelope from a non-retroviral enveloped virus (e.g., baculovirus).

### Packaging Signal

As used herein, the term “packaging signal” or “packaging sequence” refers to sequences located within the retroviral genome or a vector that are required for, or at least facilitate, insertion of the viral or vector RNA into the viral capsid or particle. The packaging signals in an RNA identify that RNA as one that is to be packaged into a virion. The term “packaging signal” is also used for convenience to refer to a vector DNA sequence that is transcribed into a functional packaging signal. Certain packaging signals may be part of a gene, but are recognized in the form of RNA, rather than as a peptide moiety of the encoded protein.

A key distinction between a packaging vector and a transgene vector is that in the packaging vector, the major packaging signal is inactivated, and, in the transgene vector, the major packaging sign al is functional. Ideally, in the packaging vector, all packaging signals would be inactivated, and, in the transgene vector, all packaging signals would be functional. However, countervailing considerations, such as maximizing viral titer, or inhibiting homologous recombination, may lend such constructs less desirable.

### Packaging System; Packaging Vectors; Packaging Cell Line

A packaging system is a vector, or a plurality of vectors, which collectively provide in expressible form all of the genetic information required to produce a virion that can encapsidate suitable RNA, transport it from the virion-producing cell, transmit it to a target cell, and, in the target cell, cause the RNA to be reverse transcribed and integrated into the host genome in a such a manner that a transgene incorporated into the aforementioned RNA can be expressed. However, the packaging system should be substantially incapable of packaging itself. Rather, it packages a separate transgene vector.

In certain embodiments of the present invention, the packaging vector will provide functional equivalents of the gag and pol genes (a “GP” vector). The env gene(s) will be provided by the envelope vector. In theory, a three vector system (“G”, “P”, and “E” vectors) is possible if one is willing to construct distinct gag and pol genes on separate vectors, and operably link them to different regulatable promoters (or one to a regulatable and the other to a constitutive promoter) such that their relative levels of expression can be adjusted appropriately.

### Envelope Proteins

The envelope proteins encoded by the packaging vector are viral proteins. An example of a non-lentiviral envelope protein is the vesicular stomatitis virus (VSV) G protein. VSV-G pseudotyped particles are rigid and can be concentrated more than 1000-fold. The vector containing an envelope protein that is different from the packaging virus genes is commonly referred to as an envelope pseudotyping vector.

Env proteins: The Env proteins of a retrovirus may be replaced with Env proteins of other retroviruses, of nonretroviral viruses, or with chimeras of these proteins with other peptides or proteins. Examples are baculovirus is Autographa californica multinuclear polyhedrosis virus (AcMNPV) envelope glycoprotein glycoprotein-64 (GP64), an envelope glycoprotein from a type D influenzae virus, an F protein for an insect virus, or a metaviridae envelope protein. In one embodiment, the glycoprotein from a type D influenzae virus is a glycoprotein-75 (GP75) protein. These envelope proteins increase the range of cells which can be transduced with retroviral derived vectors.

Chimeric Env Proteins: A chimera may be constructed of an env protein and of a ligand that binds to a specific cell surface receptor in order to target the vector to cells expressing that receptor. Examples are chimeras including FLA16 (a 6 amino acid peptide that binds integrin receptors), erythropoietin (which binds the erythropoietin receptor), human heregulin (which binds the EGF and related receptors). Alternatively, the chimera could include an antibody variable light or heavy domain, or both domains joined by suitable peptide linker (a so-called single chain antibody). Such an antibody domain could target any desired cell surface molecule, such as a tumor antigen, the human low-density lipoprotein receptor, or a determinant on human MHC Class I molecules.

Derivatized Env Proteins: Virions may be chemically, enzymatically or physically modified after production in order to alter their cell specificity. Examples of modifications include chemical or enzymatic addition of a ligand that would be recognized by a cell surface receptor (e.g., addition of lactose so that the virions will transduce human hepatoma cells which express asialoglycoprotein receptors), or incubation of the virus with a biotinylated antibody directed against the vector's Env protein, followed by addition of a streptavidin-linked ligand recognized by the cell-surface receptor. A heterobispecific antibody also could be used to link the virion's Env protein to such a ligand.

### Transgene Vectors

A transgene vector is an expression vector that bears an expressible nonretroviral gene of interest and includes at least one functional retroviral packaging signal, so that, after the transgene vector is transfected into a packaging cell line, the transgene vector is transcribed into RNA, and this RNA is packaged into an infectious viral particle. These particles, in turn, infect target cells, their RNA is reverse transcribed into DNA, and the DNA is incorporated into the host cell genome as a proviral element, thereby transmitting the gene of interest to the target cells.

As used herein, the term “transduction” refers to the delivery of a gene(s) using a viral or retroviral vector by means of infection rather than by transfection. In certain embodiments, retroviral vectors are transduced. Thus, a “transduced gene” is a gene that has been introduced into a cell via retroviral or vector infection and provirus integration. In certain embodiments, viral vectors (e.g., “transgene vectors”) transduce genes into “target cells” or host cells. The present invention encompasses transgene vectors that are suitable for use in the present invention that are include sequence that encode a gene of interest (e.g., a “marker gene” or “reporter gene,” used to indicate infection or expression of a gene).

The term “stable transduction” or “stably transduced” refers to the introduction and integration of foreign DNA into the genome of the transduced cell. The term “stable transductant” refers to a cell that has stably integrated foreign DNA into the genomic DNA.

The term “transient transduction” or “transiently transduced” refers to the introduction of foreign DNA into a cell where the foreign DNA fails to integrate into the genome of the transduced cell. The foreign DNA persists in the nucleus of the transducted cell for several days. During this time the foreign DNA is subject to the regulatory controls that govern the expression of endogenous genes in the chromosomes. The term “transient transductant” refers to cells that have taken up foreign DNA but have failed to integrate this DNA.

### Transgene

The transgene is a gene encoding a polypeptide that is foreign to the retrovirus from which the vector is primarily derived and has a useful biological activity in the organism into which it is administered (e.g., it may be a marker gene or a therapeutic gene).

One example of a transgene is a therapeutic gene. As used herein, the term “therapeutic gene” refers to a gene whose expression is desired in a cell to provide a therapeutic effect, e.g., to treat a disease.

Gene therapy may be used to successfully correct hereditary genetic errors. The molecular genetics of cystic fibrosis (CF) has been studied. Many CF patients carry a single amino acid deletion (F508) in one of the two nucleotide-binding domains in the CF transmembrane regulator (CFTR) protein. Other forms of genetic mutations in the CFTR genes have also been identified. This rich genetic information makes CF an ideal gene therapy candidate.

The target cells for CF patients are undifferentiated, proliferating and differentiated, non-proliferating lung epithelial cells. For example, both the dividing and non-dividing lung epithelial cell types can be targeted by pseudotyped retroviral vectors carrying a wild type CFTR cDNA. Recent studies suggest that gene therapy may offer great benefits to CF patients even if only partial correction of CFTR gene function is achieved.

### Selectable and Screenable Markers

A vector may contain one or more selectable or screenable markers. Such markers are typically used to determine whether the vector has been successfully introduced into a host or target cell. A selectable marker is a gene whose expression substantially affects whether a cell will survive under particular controllable conditions. A selectable marker may provide for positive selection (cells with the marker are more likely to survive), negative selection (cells with the marker are less likely to survive), or both (the choice of environmental condition dictating whether positive or negative selection occurs).

### Regulation of Gene Expression

The transgene(s) of the vector, and the marker(s) and viral genes (or replacements) of the packaging and transgene vectors, and the glycoprotein genes of the envelope vector are expressed under the control of regulatory elements.

As used herein, the term “regulatory element” refers to a genetic element that controls some aspect of the expression of nucleic acid sequences. For example, a promoter is a regulatory element that facilitates the initiation of transcription of an operably linked coding region.

Transcriptional control signals in eukaryotes comprise “promoter” elements. As used herein, the term “promoter” denotes a segment of DNA that contains sequences capable of providing promoter functions. A promoter may be a regulatable, inducible, or repressible promoter.

### Expression Vector

As used herein, the term “vector” is used in reference to nucleic acid molecules that can be used to transfer nucleic acid from one cell to another.

The term “expression vector” as used herein refers to a recombinant DNA molecule containing a desired coding sequence and appropriate nucleic acid sequences necessary for the expression of the operably linked coding sequence in a particular host. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

Pseudotyping Retroviral Vectors with Novel Envelope Glycoproteins to Enhance Target Cell Transduction.

Retroviral vector-mediated gene transfer begins with the attachment of the virion to a specific cell surface receptor. This attachment is the first step in the gene transfer process and an important factor in determining vector tropism and the range of target tissues/cell types. Vector binding is mediated by specific interactions between the envelope glycoproteins on the virion and one or more surface receptor molecules on the target cell. If this receptor molecule is absent (as when its expression is specific for certain cell types) or is variant in the binding region (such as in species other than the natural host), gene transfer may not occur. By replacing the native envelope protein with other retroviral or non-retroviral glycoproteins, a process termed “pseudotyping,” one can alter the host range of the vectors, which can result in increased transduction efficiency of desirable target cells.

To increase the in vivo gene transfer efficiency of vectors to cells and tissues for therapeutic gene delivery, the vectors may be pseudotyped with viral envelope glycoproteins from non-retroviral enveloped viruses.

GP64 is the viral binding and fusion protein of the baculovirus Autographa californica multinuclear polyhedrosis virus (AcMNPV). Envelopes from the family of related glycoproteins that includes baculovirus GP64, baculovirus F proteins, metaviridae envelopes, and the GP75 proteins of influenze D viruses (thogoto virus and dhori virus) can be used in the present invention. Examples of pseudotyped vectors can be found, for example, in U.S. Pat. Nos. 7,135,339 and 7,160,727 and in U.S. Patent Publications 2007/005929, 2005/0100890, 2005/0112098 and 2006/0093590. Other envelope that may also be used are Filovirus envelopes (e.g., Ebola, Marburg virus), Coronavirus envelopes (e.g., 229E, SARS-CoV), and Influenza A.

### Administration

The lentiviral vectors are administered to the patient so that the vectors contact cells of the patient's respiratory system. For example, the vector may be administered directly via an airway to cells of the patient's respiratory system. The vectors can be administered intranasally (e.g., nose drops) or by inhalation via the respiratory system, such as by propellant based metered dose inhalers or dry powders inhalation devices.

Formulations suitable for administration include liquid solutions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The vector can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Detergent and/or alcohol based formulations, if directly mixed with a lentivirus or retrovirus, may inactivate the virus by disrupting their lipid bilayer. Thus, in certain embodiments, formulations may exclude detergents and/or alcohols.

The vectors, alone or in combination with other suitable components, can be made into aerosol formulations to be administered via inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer. In certain embodiments, administration may be, e.g., aerosol, instillation, intratracheal, intrabronchial or bronchoscopic deposition.

In certain embodiments, the vectors may be administered in a pharmaceutical composition. Such pharmaceutical compositions may also comprise a pharmaceutically acceptable carrier and other ingredients known in the art. The pharmaceutically acceptable carriers described herein, including, but not limited to, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices. Viscoelastic gel formulations with, e.g., methylcellulose and/or carboxymethylcellulose may be beneficial (see Sinn et al., Am J Respir Cell Mol Biol, 32(5), 404-410 (2005)).

The vectors can be administered by any conventional method available for use in conjunction with pharmaceuticals, either as individual therapeutic agents or in combination with at least one additional therapeutic agent.

In certain embodiments, the vectors are administered with an agent that disrupts, e.g., transiently disrupts, tight junctions, such as EGTA (see U.S. Pat. No. 6,855,549).

The total amount of the vectors administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

The term “therapeutically effective amount”, in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat”, “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

The invention will now be illustrated by the following non-limiting Example.

### Example 1. Re-Administration of Lentiviral Vectors to Nasal Epithelia

Innate immune responses to viruses or viral vectors are a first line of defense generated within minutes to hours following viral vector administration, and contrast with the slower to develop adaptive immunity. These responses have been extensively studied with the adenoviral and AAV vectors under investigation for pulmonary gene transfer applications. Many preclinical studies and clinical trials with adenoviral vectors document increases in systemic chemokines and proinflammatory cytokines including RANTES, IP-10, MIP-2, IFN-g, TNF-a, IL-6, IL-10 and IL-12 in human and mice. A number of studies indicate that a priming dose of an adenoviral vector will elicit sufficient humoral and cell-mediated immunity to prevent pulmonary re-administration without the use of immunosuppressive agents, or the masking of vector epitopes with formulations such as pegylation. AAV vectors have also been extensively investigated in animal models and in phase I and II clinical trials for cystic fibrosis. A number of capsid serotypes have been identified and tested. In most cases, a second dose of AAV yields lower levels of pulmonary gene transfer because of neutralizing antibody responses. Pre-existing immunity against either Ad or AAV may also present a significant barrier to initial use of these vectors.

In contrast to the extensive literature regarding adenovirus and AAV, relatively little is known regarding the innate and adaptive immune responses to retrovirus-based vectors. As described herein, minimal innate and adaptive immune responses were observed following topical delivery of GP64-FIV-Luc to murine airway epithelia. A transient early increase in IL-6 and KC (IL-8 ortholog) release occurred following delivery of ˜1×10⁷ transducing units of FIV, titer matched Ad5, or vehicle alone. These data suggest that vehicle impurities and/or simple physical irritation of the respiratory mucosa are sufficient to elicit transient cytokine release. Importantly, this cytokine response does not preclude persistent transgene expression following lentiviral vector transduction.

The GP64 pseudotyped FIV vector efficiently transduced nasal epithelia and persistently expressed a transgene. Additive increases in transgene expression were demonstrated with repeat dosing. This increase in expression may represent both an increase in the percentage of cells expressing a transgene (see FIG. 2) and an increase in the number of transgene copies/cell. The route of vector administration may influence the subsequent development of immune responses. The results in the nasal airways indicate that mucosal application of a lentivirus vector is less immunostimulatory than systemic delivery.

For genetic diseases, the expression of a therapeutic protein over the life of the affected individual is a goal. Therefore, repeated administration of viral vectors may be necessary, and this presents unique limitations for each vector system. The re-administration of lentiviral vectors to the respiratory tract offers the possibility to increase the overall transduction efficiency. For example, sequential vector administration to individual lung lobes may be advantageous for practical and safety reasons. In addition, the ability to successfully re-dose provides a therapeutic option should expression wane over time. These findings have important implications for the translation of lentiviral gene transfer technology into tools for investigating respiratory cell biology and the development of pulmonary disease therapies in large animal models and human trials.

For many envisioned applications of lentiviral vectors as tools in respiratory biology and therapeutic gene delivery, the efficiency of gene transfer needs to be improved. The efficacy of repeatedly administering lentiviral vectors to the airways is described herein. Using quantitative bioluminescent imaging, it was found that consecutive daily dosing achieved a linear increase in gene expression and greatly increased the number of epithelial cells targeted. Surprisingly, reporter gene expression also increased additively following each of 7 doses of FIV delivered over consecutive weeks (1 dose/week), without the development of systemic or local neutralizing antibodies. This approach enhanced expression of both reporter and therapeutic transgenes. Transduction efficiency achieved following a single dose of FIV expressing mouse erythropoietin was insufficient to increase hematocrit, whereas 7 consecutive daily doses significantly increased hematocrit. These unexpected results contrast strikingly with findings reported for adenoviral vectors. While prolonged gene expression has been observed in vivo following a single dose of viral vector, depending on the application, repeated vector administration will be beneficial to achieve stable, therapeutic gene expression. Accordingly, described herein are results demonstrating that lentivirus vectors can be readministered to nasal epithelia without blocking immune responses.

Several issues limit the application of gene transfer as a tool for pulmonary cell biology studies and impede its translational utility for treating diseases of respiratory epithelia. A limitation for many vector systems is the inability to re-administer as transgene expression wanes. Mucosal innate and adaptive immune responses against the vector or vector-encoded proteins represent a significant impediment to clinical applications and are well documented for viral vectors such as adenovirus (Ad) and adeno-associated virus (AAV). Indeed, a driving force behind the development of helper-dependent adenoviral vectors and the search for alternative AAV vector capsids has been avoidance of adaptive immune responses. An alternative strategy is the use of integrating viral vectors of the retrovirus family.

As described herein, it was investigated whether it is possible to repeat lentivirus vector administration to the respiratory tract and increase gene transfer. A GP64 pseudotyped FIV was repeatedly delivered to murine nasal epithelia. Transduction efficiency, persistence of expression, and host responses were investigated. The successful re-administration of reporter and therapeutic transgenes to respiratory epithelia without the development of mucosal inhibitory antibodies is reported herein. These novel findings in the nasal epithelia have implications for the development of gene transfer strategies to study airway biology and to treat genetic and acquired disorders of the respiratory system.

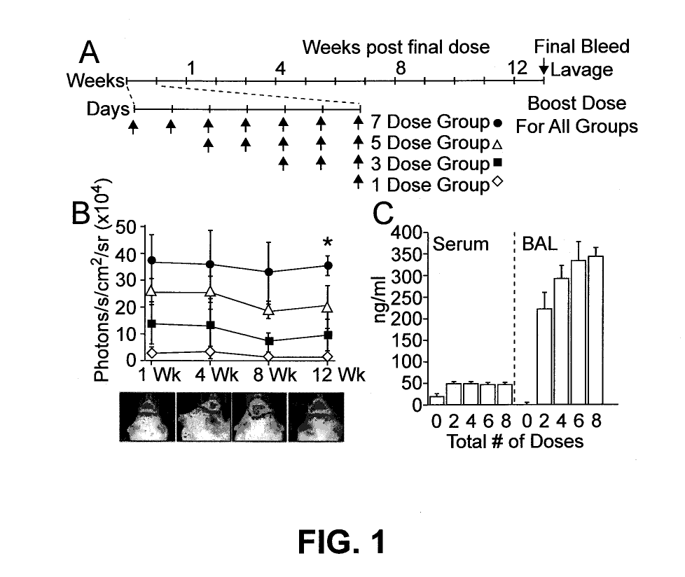

Thus, to investigate expression-enhancing benefits of acute lentiviral vector re-administration to nasal epithelia, Luc was used as a sensitive and easily quantified reporter gene. In this experimental protocol (FIG. 1A), 4 groups of mice received 1, 3, 5, or 7 total doses of GP64-FIV-Luc over the same number of consecutive days. A fifth group received no treatment and served as a baseline of background luminescence. Background luminescence may vary slightly from day to day, thus naive mice were included in every imaging session, and baseline values were always subtracted from the experimental values. One week following the final dose, mice underwent bioluminescence imaging, and animals were imaged again 4, 8, and 12 weeks following the final dose. After the final imaging, mice received a vector booster dose and sera and bronchoalveolar lavage (BAL) were collected. As shown in FIG. 1B, there was a linear increase in Luc expression from 1 to 7 doses observed at 1 week post delivery and expression remained stable over the duration of the experiment. These data confirm that acute repeat administration is an effective means to improve the gene transfer efficiency.

The total concentrations of anti-GP64 IgG antibodies in sera and BAL were determined one week following a boost dose. Low level IgG anti-GP64 antibodies (˜45 ng/ml) were observed in the sera for all groups (FIG. 1C) and higher levels of IgG anti-GP64 antibodies (˜200-350 ng/ml) were observed in the BAL (FIG. 1C). Importantly, the levels of inactivating antibodies in BAL were below the limit of detection. These data indicate that while an adaptive immune response is mounted against the vector, it is insufficient to block gene transfer.

In parallel experiments, GP64-FIV expressing nuclear targeted β-galactosidase ((3-gal) was delivered to a separate cohort of mice following the same experimental timeline described in FIG. 1A. One month following vector delivery, coronal sections through the mouse muzzle were obtained as shown schematically (FIG. 2A). Tissue section levels were arbitrarily assigned and were matched between mice based on morphologic landmarks (1 level=30 microns). Low power digital photographs (FIG. 2B) were taken of the olfactory (FIG. 2C) and respiratory (FIG. 2D) epithelia and the percentage of transduced cells determined. Within the olfactory epithelia, a greater percentage of β-gal positive cells was observed rostrally (level 100) compared to caudally (level 200) (FIG. 2E). For respiratory epithelia, the greatest percentage of β-gal positive cells was detected at a mid point (level 150) (FIG. 2F). By averaging the total percent positive cells across all levels, a dose-dependent increase in expression was observed from <1% for a single dose to ˜10% for seven doses (FIG. 2G). Further, no preferential GP64-mediated transduction of respiratory or olfactory epithelium was observed.

The innate immune response following delivery of a single dose of GP64-FIV, Ad5, or vehicle was examined by measuring levels of 14 cytokines in nasal lavage at 4, 24, and 72 hrs after delivery (Table 1). Most cytokines increased slightly (<50 pg/ml) at 4 hrs and returned to naive levels by 24 or 72 hrs. Two cytokines, KC and IL-6, increased the most at 4 hrs (2,000-3,000 pg/ml), but returned to naive levels by 24 hrs. In addition, no differences were observed between GP64-FIV, Ad5, or vehicle. These data suggest that vehicle administration, in combination with the simple act of mucosal stimulation, is sufficient to elicit a response.

TABLE 1

| | | | IL-12 | | | RAN- | GM- | | | IL-12 | | | | | | |
|---|---|---|---|---|---|---|---|---|---|---|---|---|---|---|---|---|
| | | Hrs | (p40) | KC | MCP-1 | TES | CSF | IFN-γ | IL-10 | (p70) | IL-6 | IL-1β | IL-2 | IL-4 | IL-5 | TNF-α |
| NWF^{c} | FIV | 4 | <3^{a} | 2963 ± 835 | 43 ± 3 | 11 ± 1 | 36 ± 15 | 15 ± 2 | 10 ± 6 | 49 ± 10 | 1764 ± 671 | 21 ± 4 | 12 ± 2 | 14 ± 2 | 15 ± 2 | 43 ± 15 |
| | | 24 | <3 | 357 ± 70 | 20 ± 11 | 11 ± 1 | 9 ± 5 | 7 ± 2 | <3 | <3 | 49 ± 49 | <3 | 7 ± 1 | 10 ± 1 | 11 ± 1 | 11 ± 1 |
| | | 72 | <3 | 261 ± 24 | 16 ± 9 | 10 ± 1 | 12 ± 4 | 8 ± 1 | <3 | <3 | 42 ± 10 | <3 | 8 ± 2 | 10 ± 1 | 9 ± 3 | 12 ± 1 |
| | Ad | 4 | <3 | 3318 ± 630 | 26 ± 8 | 11 ± 1 | 35 ± 4 | 14 ± 1 | 5 ± 4 | 49 ± 7 | 2355 ± 572 | 21 ± 5 | 10 ± 1 | 13 ± 1 | 14 ± 1 | 36 ± 6 |
| | | 24 | <3 | 365 ± 27 | 35 ± 3 | 10 ± 1 | 7 ± 3 | 9 ± 1 | <3 | <3 | 60 ± 16 | <3 | 8 ± 1 | 10 ± 1 | 5 ± 2 | 13 ± 1 |
| | | 72 | <3 | 254 ± 39 | 27 ± 9 | 10 ± 1 | 7 ± 4 | 10 ± 1 | <3 | 9 ± 5 | 42 ± 3 | <3 | 5 ± 2 | 10 ± 1 | 12 ± 2 | 15 ± 2 |
| | Veh^{b} | 4 | <3 | 3243 ± 288 | 37 ± 4 | 10 ± 1 | 32 ± 8 | 13 ± 1 | 6 ± 2 | 45 ± 6 | 1800 ± 487 | 22 ± 4 | 11 ± 1 | 13 ± 1 | 15 ± 1 | 36 ± 4 |
| | | 24 | <3 | 403 ± 41 | 32 ± 4 | 12 ± 1 | 11 ± 3 | 9 ± 1 | <3 | 5 ± 3 | 162 ± 44 | 5 ± 2 | 10 ± 1 | 9 ± 1 | 8 ± 4 | 17 ± 1 |
| | | 72 | <3 | 326 ± 23 | 26 ± 12 | 12 ± 1 | 20 ± 4 | 11 ± 1 | <3 | 5 ± 3 | 86 ± 24 | 11 ± 1 | 9 ± 1 | 10 ± 1 | 11 ± 3 | 22 ± 2 |
| | Naïve | 4 | <3 | 707 ± 47 | 28 ± 9 | 13 ± 1 | 14 ± 4 | 10 ± 1 | <3 | 6 ± 5 | 141 ± 54 | 13 ± 1 | 9 ± 1 | 11 ± 1 | 12 ± 1 | 25 ± 2 |
| | | 24 | <3 | 448 ± 67 | 12 ± 7 | 12 ± 1 | 10 ± 4 | 9 ± 1 | <3 | <3 | 42 ± 8 | 5 ± 2 | 9 ± 1 | 10 ± 1 | 8 ± 2 | 16 ± 1 |
| | | 72 | <3 | 217 ± 42 | 12 ± 10 | 10 ± 1 | 9 ± 4 | 9 ± 1 | <3 | <3 | 34 ± 9 | <3 | 8 ± 1 | 9 ± 1 | 12 ± 2 | 13 ± 1 |
| Serum | FIV | 24 | 6 ± 1 | 100 ± 11 | 15 ± 4 | 20 ± 1 | 15 ± 5 | 13 ± 9 | 8 ± 5 | 28 ± 17 | 11 ± 7 | 42 ± 24 | 7 ± 4 | 4 ± 3 | 5 ± 3 | 2 ± 1 |
| | Ad | 24 | 7 ± 2 | 91 ± 12 | 11 ± 1 | 20 ± 1 | 16 ± 6 | 15 ± 6 | 12 ± 6 | 36 ± 19 | 11 ± 4 | 40 ± 20 | 6 ± 3 | 5 ± 3 | 4 ± 1 | 2 ± 1 |
| | Veh | 24 | 29 ± 24 | 65 ± 14 | 8 ± 1 | 18 ± 2 | 10 ± 1 | 6 ± 2 | 4 ± 1 | 11 ± 3 | 4 ± 1 | 14 ± 5 | 3 ± 1 | 1 ± 1 | 3 ± 1 | 2 ± 1 |
| | Naïve | 24 | 5 ± 1 | 41 ± 11 | 13 ± 3 | 21 ± 3 | 11 ± 2 | 12 ± 5 | 7 ± 2 | 17 ± 4 | 9 ± 3 | 25 ± 9 | 2 ± 1 | 3 ± 1 | 3 ± 1 | 2 ± 1 |

^{a}All numerical data are average (pg/ml) ± standard deviation, n = 4 animals/timepoint
^{b}Vehicle composed of 50:50 mix of 1% methylcellulose and tris-lactose buffer
^{c}Nasal wash fluid

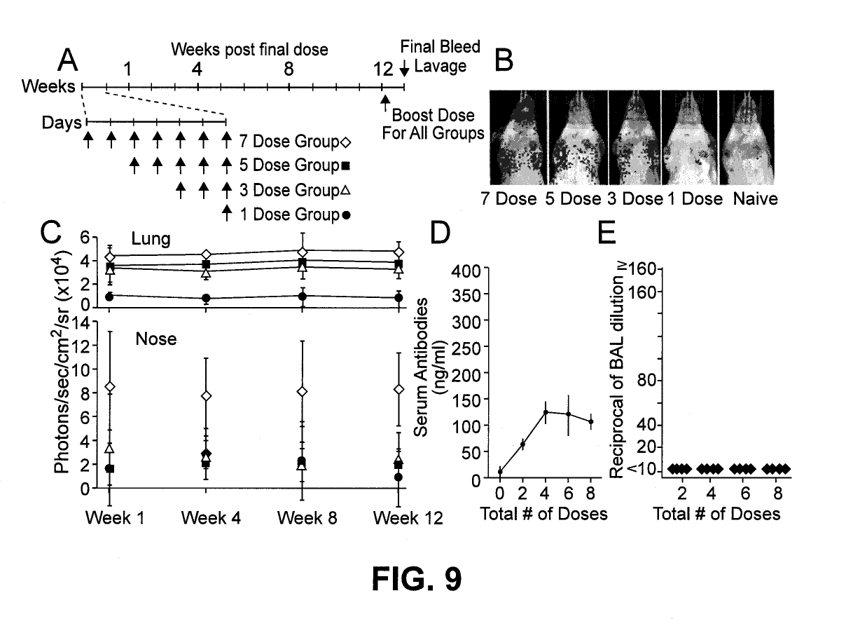

In addition to acute re-administration to nasal epithelia, successive doses of GP64-FIV-Luc were delivered to the intrapulmonary airways via tracheal instillation (FIG. 9). As with nasal instillation, Luc expression increased following repeated dosing. Interestingly, both lung and nasal Luc expression were observed following direct tracheal instillation, possibly due to mucociliary clearance of vector. Total serum IgG anti-GP64 antibodies increased slightly following tracheal delivery as compared to nasal instillation; however, inactivating antibodies were below the limit of detection.

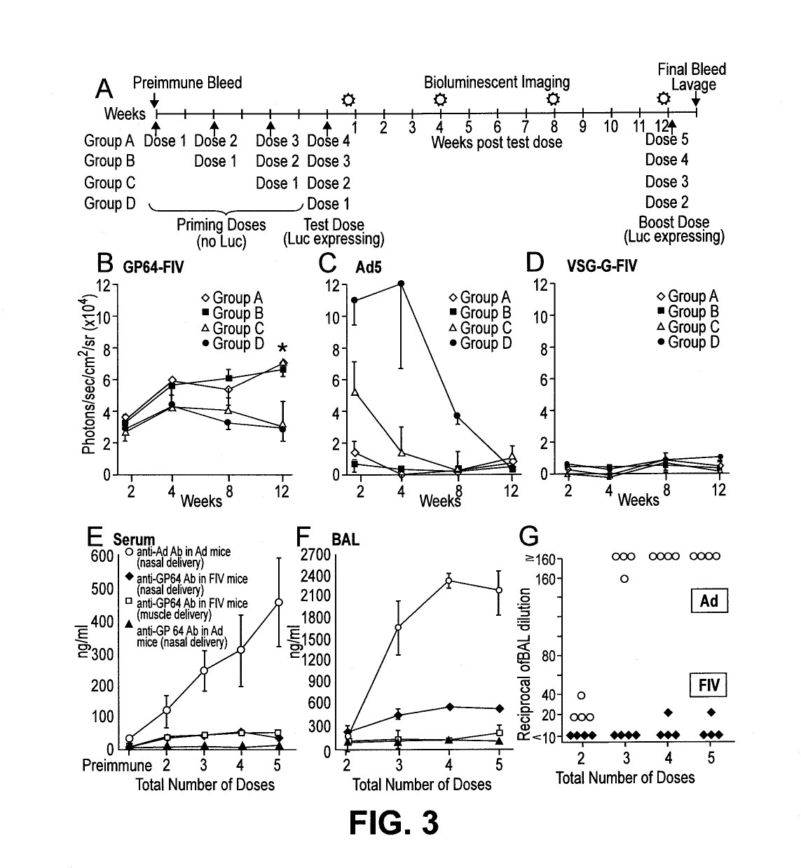

To investigate the possibility of increasing the time interval between vector doses, priming doses of GP64-FIV, adenovirus serotype 5 (Ad5), or Vesicular stomatitis virus glycoprotein (VSV-G) pseudotyped FIV vectors were delivered to nasal epithelia followed by a test dose. The priming dose(s) of FIV carried a firefly Luc transgene without a promoter. No luciferase expression was observed following the delivery of this vector either in vitro or in vivo. The test dose was the identical lentiviral vector, with an RSV promoter driving Luc. Mice received 3, 2, 1, or 0 priming doses at two week intervals (Groups A, B, C, and D, respectively) followed by a test dose (FIG. 3A). Two weeks was chosen as sufficient time for the development of adaptive immune responses. Following the test dose, mice were imaged at 4 day, 4 week, 8 week, and 12 week time points (FIG. 3A). At the 12 week time point, a boost dose was given. One week later, serum and BAL were collected. Importantly, priming doses of GP64-FIV did not result in a loss of expression from the test dose (FIG. 3B). Unexpectedly, animals that received 2 or 3 priming doses had higher Luc expression after 12 weeks than animals receiving 0 or 1 priming dose (FIG. 3B). As expected, in mice that received Ad-Empty (priming dose) followed by Ad-Luc (test dose), expression from the test dose was significantly attenuated after a single priming dose (FIG. 3C). No expression was observed in mice that received VSV-G pseudotyped FIV (FIG. 3D), consistent with previous observations that this vector poorly transduces polarized epithelia in the absence of agents that disrupt tight junctions.

IgG antibodies against Ad or GP64 were measured in serum (FIG. 3E) or BAL (FIG. 3F) collected 1 week after the boost dose at the end of the delivery protocol (FIG. 3A). As expected, a dose-dependent increase in anti-Ad antibodies in sera was observed following Ad vector intra-nasal (I.N.) instillation (FIG. 3E). In contrast, serum levels of total anti-GP64 IgG antibodies following I.N. or intra-muscular (I.M.) GP64-FIV administration plateaued after 3 doses (FIG. 3E). The findings for total anti-Ad or GP64 IgG antibody production in BAL mirrored the sera, with the exception that no antibodies were observed in BAL following I.M. GP64-FIV (FIG. 3F). In mice that received Ad vector, no detectable anti-GP64 IgG antibodies were found in sera (FIG. 3E) or BAL (FIG. 3F). The attenuated expression of Ad correlated with production of neutralizing antibodies following repeated dosing (FIG. 3G). In contrast, only low level neutralizing antibody production was observed following lentiviral vector delivery, and neutralizing antibody levels did not increase significantly with repeated doses (FIG. 3G).

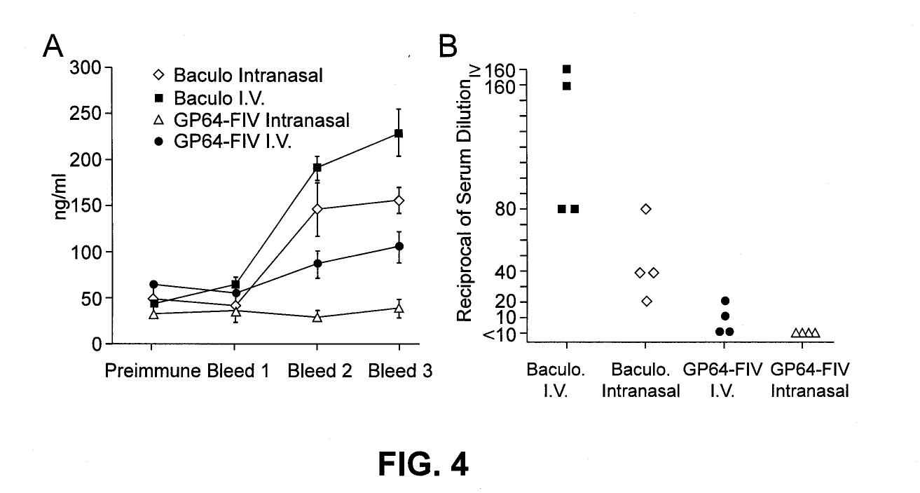

The GP64 envelope glycoprotein used to pseudotype FIV is the same in amino acid sequence to the Autographa Californica baculoviral envelope, which can elicit a strong immune response. Indeed, baculoviral expressed GP64-fusion proteins provide tools for antibody generation. However, baculovirus envelope glycoprotein produced in insect cells will display different glycosylation patterns than GP64-FIV produced in human cells. 3 doses of baculovirus or GP64-FIV were delivered via I.N. or tail vein (I.V.) routes at two week intervals. The delivered doses (˜9.1×10⁶ TU of GP64-FIV or ˜3.0×10⁶ pfu of baculovirus) were matched for total GP64 protein as determined by immunoblot. The greatest serum IgG anti-GP64 antibody levels were observed in mice receiving I.V. or I.N. baculovirus delivery (FIG. 4A). The prevalence of neutralizing antibodies (FIG. 4B) was consistent with the total serum anti-GP64 antibodies. These data suggest that baculovirus associated immunogenicity is likely due to a combination of the delivery route, the GP64 glycosylation pattern, and other adjuvants missing from lentiviral vector preparations.

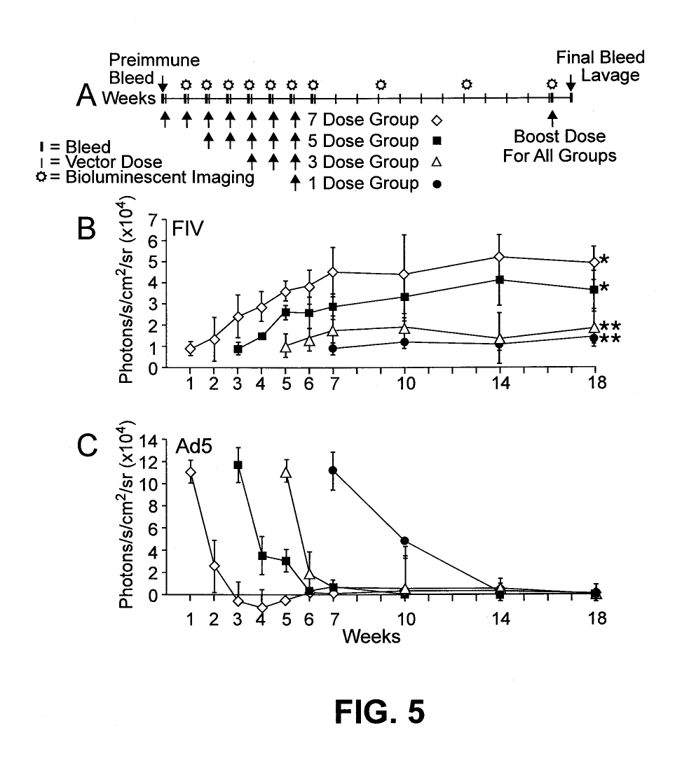

To investigate further the relationship between dosing interval and sustained increases in transgene expression, 1, 3, 5, or 7 doses of vector were delivered over the same number of consecutive weeks (1 dose/week) (FIG. 5A). Between each dose, mice were imaged and sera collected. Of note, with weekly administration of GP64-FIV-Luc, a linear increase in reporter gene expression was again observed (FIG. 5B). Furthermore, expression was stable for the duration of the experiment. In contrast, with weekly administration of Ad5-Luc, expression quickly dropped to naive levels (FIG. 5C).

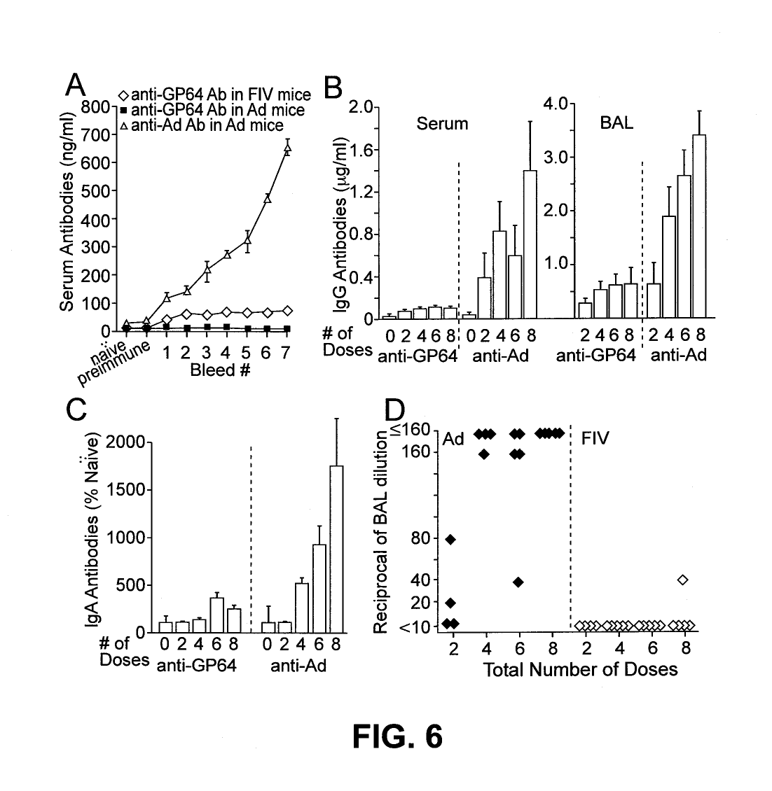

Total IgG serum antibodies generated against Ad5 or GP64 were quantified by ELISA following each weekly vector dose (FIG. 6A). As expected, anti-Ad5 antibodies increased with successive doses. In contrast, mice receiving repeated administration of GP64-FIV-Luc generated anti-GP64 antibodies that plateaued after the second dose. As a negative control, anti-GP64 antibodies were measured following Ad5-Luc and observed no antibodies. The total serum and BAL (FIG. 6B) IgG antibodies against Ad5 or GP64 were quantified 1 week following a boost dose of the appropriate vector at the end of the 19 week experiment. For Ad5-Luc, a dose-dependent increase in IgG antibody production was observed in both sera and BAL (FIG. 6B). In contrast, mice that received GP64-FIV-Luc repeatedly generated low levels of anti-GP64 antibodies that reached a plateau following 4 doses in both sera and BAL (FIG. 6B). Again, no anti-GP64 antibodies were detected in either the sera or BAL following repeated doses of Ad5-Luc. Similarly, little evidence of anti-GP64 IgA antibody response was present in BAL after single or multiples dose (FIG. 6C). This contrasted with the robust IgA responses elicited by Ad (FIG. 6C). Neutralizing antibodies against Ad5-Luc were detected in the BAL in 2 of 4 mice after 2 doses and all (14 of 14 total) mice after 4, 6, or 8 doses (FIG. 6D). Conversely, no inactivating antibodies to GP64-FIV-Luc were detected, with the exception of 1 of 5 mice after 8 doses (FIG. 6D). These encouraging results suggest that GP64-FIV-based vectors are well suited for repeat administration to nasal epithelia.

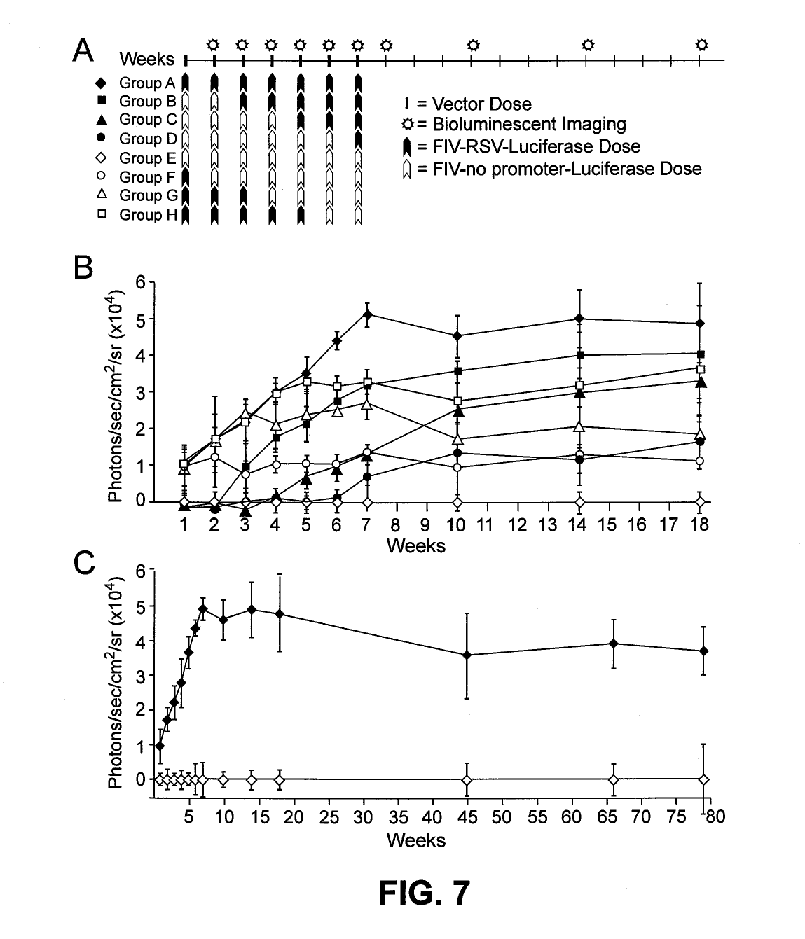

An additional experiment was performed to investigate how previous vector exposure affects the success of repeat administration with longer dosing intervals. Eight groups of mice (5 per group) received 7 doses of GP64-FIV-Luc (FIG. 7A). However, the vector either contained an RSV internal promoter driving Luc (filled arrows) or lacked an internal promoter (open arrows). Again, mice that received 7 doses of FIV-RSV-Luc displayed a linear increase in reporter gene expression (FIG. 7B). Animals receiving 7 doses of FIV-no promoter-Luc displayed no expression above naive controls (not shown), while those pre-treated with 2 or 4 doses of FIV-no promoter-Luc displayed linear increases in expression with each successive dose of FIV-RSV-Luc. Likewise, subsequent applications of 6, 4, or 2 doses of FIV-no promoter-Luc conferred stable Luc expression over the 3 months following the final dose. A trend was noted for mice pre-dosed with FIV-no promoter-Luc to exhibit higher Luc expression at the conclusion of the experiment than animals receiving post-dosing of FIV-no promoter-Luc (compare closed squares, triangles, and circles to open squares, triangles and circles, respectively). Long-term stable expression lasting >78 weeks (1.5 years) was documented in mice that received 7 doses of GP64-FIV-Luc (FIG. 7C).

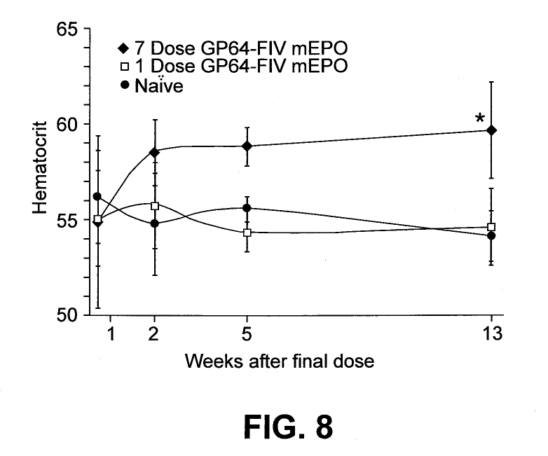

To investigate the potential for enhancing secreted protein expression following topical administration of lentiviral vector to nasal epithelia, murine erythropoietin (mEPO) was selected as a transgene. Recombinant EPO is used clinically to treat anemia of chronic disease. Airway delivered mEPO expressed from an Ad vector was previously demonstrated to increase hematocrit. Here, 7 doses of GP64-FIV-mEPO delivered over 7 consecutive days (1 dose/day) was compared to a single dose (FIG. 8). By 2 weeks post-delivery, functional mEPO expression was observed from the 7 dose group as evidenced by a significant increase in hematocrit. The hematocrit increase persisted for 13 weeks, the last time point tested. The hematocrit of the 1 dose group was not statistically different than the naive control group. These results further demonstrate that repeated vector administration is feasible and may be needed to achieve desired expression levels for some transgenes.

### Materials and Methods

Vector Production.

The FIV vector system utilized in this study (see Johnston et al., J Virol, 73, 4991-5000 (1999) and Wang et al., J Clin Invest, 104, R55-62 (1999)) expressed either mouse erythropoietin (mEPO), nuclear-targeted β-gal, or firefly Luc. Pseudotyped FIV vector particles were generated by transient transfection, concentrated 250-fold by centrifugation, and titered using real-time PCR as previously described (Sinn et al., Hum Gen Ther, 18, 1244-1252 (2007)). Mouse erythropoietin cDNA was obtained from Open Biosystems (Clone ID, 8734014; accession number, BC119265), sequence confirmed, and cloned into the FIV3.3RSV backbone (Sinn et al., Hum Gen Ther, 18, 1244-1252 (2007)).

In Vivo Viral Vector Administration.

Female, 6-10 week old, 18-22 g Balb/c mice were used in this study (Harlan; Indianapolis, Ind.). Approximately 1.25×10⁷ transducing units (TU) of FIV vector in a 50 μl volume was delivered to the nasal epithelia via direct instillation. Adenoviral vector was delivered at 1.25×10⁷ PFU in a 50 μl volume. This dose was constant for each vector administration and for each protocol. Vector was formulated with 1% methylcellulose as previously described (Sinn et al., American J Resp Cell Mot Biol, 32, 404-410 (2005)). An endotoxin assay revealed detectable levels of endotoxin (<100 endotoxin units) in the delivered volume of vehicle.

Bioluminescence Imaging. At the time-points indicated, animals were injected intraperitoneally with 100 μl/10 g body weight of D-luciferin (15 mg/ml in PBS, Xenogen, Alemeda, Calif.) using a 25-gauge needle. Approximately 5 min after luciferin injection, mice were placed in the imaging cabinet, anesthetized with 1-3% isoflurane, and imaged using the Xenogen IVIS CCD camera. Imaging data were analyzed and signal intensity quantified using Xenogen Living Image software.

GP64 Sandwich ELISA.

A 96-well microtiter plate was coated with GP64 capture antibody (470 μg/μL) and incubated overnight at 4° C. Blocking solution (1×PBS, 5% FBS) was applied for 3 hrs at room temperature without removing the capture antibody. The plate was washed with 1×PBS to remove unbound capture antibody. Standard antigen dilutions were made using purified anti-GP64 monoclonal antibody (eBioscience, cat#14-6995-81). Serum samples and antigen standards dilutions were prepared using blocking buffer and 0.0005% Tween-20. Dilutions for the sample serum and the standard-antigen were added to their respective wells in 100 μL aliquots. The plate was again incubated overnight at 4° C. and washed with 1×PBS. Goat anti-mouse IgG, HRP conjugated secondary antibody (Pierce, #31432) or goat anti-mouse IgA, HRP conjugated secondary antibody (Innovative Research Inc., #IGA-90P) was added and the plate was incubated at room temperature for 90 min, washed once again with 1×PBS, and dried. HRP substrate solution was added and absorbance was measured at 405 nm. Recombinant GP64 was used to generate a standard curve. The GP64 envelope glycoprotein is the primary protein displayed on the surface of pseudotyped virions.

Adenovirus ELISA (Indirect ELISA).

A 96-well microtiter plate was coated with Ad5 vector and incubated overnight at 4° C. Sample dilutions were made with PBS, 0.01% Tween-20. The plate was washed and appropriate standard and sample dilutions added. The plate was again incubated at 4° C. overnight. Blocking solution was added and incubated for 3 hrs. Unbound capture antibody was removed by washing with PBS, 0.01% Tween-20. Plasma and standard dilutions were added and the plate was washed once again with wash buffer (PBS, 0.01% Tween-20). Secondary antibody, HRP conjugated goat anti-mouse IgG or IgA, was added at predetermined dilution and incubated for 1 hr at room temperature followed by a final rinse and addition of the substrate for visualization. Absorbance was measured at 405 nm.

Neutralizing Antibody Assay.

The neutralizing antibody assay was adapted from a previously described protocol (Stein et al., Gene Ther, 5, 431-439 (1998)). Briefly, HT1080 cells were seeded at a density of 3×10⁶ cells/well in a 12-well plate. Dilutions were prepared for each serum or BAL sample ranging from 1:10-1:160 in serum free media. Each plate included a serum free media only control. GP64-FIV-Luc or Ad-Luc was incubated with sample dilutions prior to delivery to cells at ˜1 MOI. Media was changed after 24 hrs and lysates were collected after 96 hrs. The dilution of sera or BAL sufficient to neutralize 50% of viral vector mediated Luc expression was determined by Luc assay (Promega E-1501).

Histochemical Analysis.

Three weeks after vector delivery, heads were removed, fixed, X-gal stained, decalcified (Luna, Histopathologic Methods and Color Atlas of Special Stains and Tissue Artifacts. Johnson Printers, Downers Grove, Ill. (1992)), paraffin embedded, sectioned, and counterstained with nuclear fast red per standard techniques. 8 mm thick coronal sections were collected at 200 mm intervals. Sections were collected at arbitrary levels 100, 125, 150, 175, and 200. Stereology was performed using basic methods as previously described (Cruz-Orive et al., J Microsc, 122, 235-257 (1981)). The respiratory and olfactory epithelial areas were examined by capturing all microscopic images at 20× magnification with an Olympus DP70 digital camera and analyzed using Image-Pro Plus version 4.1 computer software (Media Cybernetics, Inc., Silver Springs, Md.). Areas of epithelium where the apical membrane reached the airway lumen were traced and the number of β-gal positive cells calculated and compared to the total number of cells within the traced area. Calculations were derived from images taken from 4 mice, and 4 epithelial areas were examined from each animal. The epithelial areas were collected ˜3.0-3.4 mm caudal from nose. Images were coded and counted by an observer blinded to this code. Measurements were counted twice independently with reproducible results.

Cytokine Assays.

At the appropriate end point of each experiment, mice were euthanized by carbon dioxide overdose. After euthanasia, the nasal cavity was washed with 200 μl saline (in 20 ml increments; 100 μl per nares) as previously described (Lu et al., FEMS Microbiol Lett, 265, 141-150 (2006) and Lu et al., Comp Med, 57, 349-354 (2007)). Saline was administered in the nares, and the nasal wash fluid was collected with a pipetter fitted with a 20 μl plastic tip, pooled, and centrifuged in a microfuge at 2,300×g for 10 minutes to pellet cells and debris. The nasal wash fluid supernatant was removed and stored at −80° C. without inhibitors. As described previously (Lu et al., FEMS Microbiol Lett, 265, 141-150 (2006) and Lu et al., Comp Med, 57, 349-354 (2007)), the concentrations (pg/ml) of 14 cytokines using a multiplex fluorescent bead-based immunoassay (Kit 48-004, Upstate Biotechnology, Lake Placid, N.Y.) were measured. Samples were incubated with anti-mouse multicytokine beads at 4° C. for 18 hrs. Unbound material was removed by filtration. Antimouse multicytokine biotin reporter was added, and reactions were incubated at room temperature for 1.5 hrs in the dark. Streptavidin-phycoerythrin then was added, and plates were incubated at room temperature for 30 min. Stop solution was added, and the plates were read in the plate reader (model 100 IS, Luminex, Austin).

Hematocrit Measurements.

Blood was collected in heparinized capillary tubes following facial vein puncture using Goldenrod lancets (MEDIpoint, Inc). The collected blood was spun in a microhematocrit centrifuge (No. 15401-628; VWR Scientific, Buffalo Grove, Ill.) for 2 min to separate plasma from red blood cells. The percentage red blood cells was measured with a metric ruler.

Statistics.

Unless otherwise noted, all numerical data are presented as the mean±standard deviation.

Please also refer to Sinn et al., Journal of Virology, 82(21), 10684-10692 (2008) for a description of experimental details, results and discussion.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for treating a patient, comprising administering a lentiviral vector that comprises a nucleotide sequence encoding a therapeutic protein to a tissue of the respiratory system of the patient, wherein the administration comprises administering the lentiviral vector in at least two consecutive dosages, wherein two consecutive dosages of the administration are separated by an interval of more than one day.

2. The method of claim 1, wherein the lentiviral vector is a human immunodeficiency viral vector, a visna-maedi virus viral vector, a caprine arthritis-encephalitis virus viral vector, an equine infectious anemia virus viral vector, a feline immunodeficiency virus (FIV) viral vector; bovine immune deficiency virus (BIV) viral vector, a simian immunodeficiency virus (SIV) viral vector, a murine Moloney leukemia virus viral vector, a foamy virus viral vector, or an avian leukosis virus viral vector.

3. The method of claim 2, wherein the lentiviral vector is a FIV viral vector.

4. The method of claim 1, wherein the lentiviral vector is pseudotyped with an envelope glycoprotein.

5. The method of claim 4, wherein the lentiviral vector is pseudotyped with a filovirus, coronavirus, or influenza envelope glycoprotein.

6. The method of claim 4, wherein the envelope glycoprotein is glycoprotein-64 (GP64).

7. The method of claim 6, wherein the envelope glycoprotein is an Autographa californica multinuclear polyhedrosis virus (AcMNPV) glycoprotein.

8. The method of claim 1, wherein the therapeutic protein is cystic fibrosis transmembrane regulator protein (CFTR), Alpha 1 antitrypsin, ATP-binding cassette A3 protein (ABCA3), surfactant protein B (SFTPB) or surfactant protein C (SFTPC).

9. The method of claim 8, wherein the therapeutic protein is CFTR.

10. The method of claim 1, wherein the tissue of the respiratory system comprises airway epithelial cells.

11. The method of claim 1, wherein the tissue of the respiratory system is lung tissue, nasal tissue, tracheal tissue, bronchial tissue, bronchiolar or alveolar epithelial cell tissue.

12. The method of claim 1, wherein the administration comprises administering at least three doses of the lentiviral vector.

13. The method of claim 12, wherein the administration comprises administering at least five doses of the lentiviral vector.

14. The method of claim 13, wherein the administration comprises administering at least ten doses of the lentiviral vector.

15. The method of claim 1, wherein the at least two consecutive dosages of the administration are separated by an interval of about one week.

16. The method of claim 1, wherein the at least two consecutive dosages of the administration are separated by an interval of about one month.

17. The method of claim 1, wherein the administration is via aerosol, dry powder, bronchoscopic instillation, or intra-airway aerosol.

18. The method of claim 1, wherein the patient has been diagnosed with cystic fibrosis.

19. The method of claim 1, wherein the patient is a human patient.
